(19) United States
(12) Patent Application Publication
(10) Pub. No.: US 2010/0030284 A1
(43) Pub. Date: Feb. 4, 2010

(54) COATED TEXTILES

(75) Inventors: Niels A. Abt, Winterhur (DE); Guido Casutt, Sulz-Rickenbach (CH)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: ZIMMER GMBH, Winterthur (CH)

(21) Appl. No.: 12/158,894

(22) PCT Filed: Dec. 21, 2006
(86) PCT No.: PCT/EP2006/012373

§ 371 Date: Jun. 30, 2009

(30) Foreign Application Priority Data
Dec. 23, 2005 (EP) 05112978.1

(51) Int. Cl. A61B 17/58; B32B 5/02; B32B 27/02; B05D 3/02; B32B 1/06; B32B 1/08

(52) U.S. Cl. 606/86 R; 442/59; 442/164; 442/166; 442/170; 442/172; 427/374.1; 428/35.2; 428/36.91

(57) ABSTRACT

A textile material (21, 21′) comprising a textile substrate (22, 22′) has, at least on part of the surface of the textile substrate (22, 22′), a coating (24, 24′) that contains at least one compound chosen from the group composed of hydrogels, except keratin hydrogels, of polyurethanes, polyvinyl chloride, polytetrafluoroethylene and any desired combinations of two or more of the aforementioned compounds. This textile material (21, 21′) can be used particularly as a substance for an artificial intervertebral disc (16″, 16′″), as a replacement for the nucleus pulposus (16′, 16″), cage (16, 16′), vertebral body implant (16″″), articular surface substitute, or as a tube (18, 18′, 18′, 18″) in vertebroplasty or nucleoplasty.

The present invention relates to a textile material comprising a textile substrate, with a coating being provided at least on a part of the surface of the textile substrate. The present invention furthermore relates to a method for the manufacture of such a textile material, to an article, in particular to an implant, and to the use of the article.

Textile materials are used in many technical fields, for example, in automotive construction, in the clothing industry, in the construction industry and in the medical field. In this connection, the technical application properties of the textile materials can be adapted to the specific demands of the later use by selection of suitable textile fiber materials, the addition of suitable auxiliary materials and the application of a coating of suitable materials. It is in particular possible to manufacture biocompatible or resorbable textiles by the use of corresponding textile fiber materials. For this reason, textile materials today are in particular also used in a versatile manner in the medical field, for example as a material for hygiene articles, filters, bandages, carrier and support materials, surgical suture materials and implants.

Implants from textile materials have, for example, proved themselves as artificial intervertebral disks and as vertebral body implants. A human spine comprises 23 intervertebral disks which are disposed like cushions between two respective adjacent vertebral bodies consisting of cortical spongious bone. In macroscopic terms, each intervertebral disk is composed of three different components which together form a functional unit, namely a semigelatinous nucleus (nucleus pulposus) disposed at the center of the intervertebral disk, a fibrocartilage ring surrounding the nucleus (anulus fibrosus) as well as two end plates which tightly contact the adjacent vertebral bodies, are arranged in each case on the top surface or base surface of the nucleus or anulus fibrosus and consist of hyaline cartilage. In this connection, the end plates form a transition zone between the intervertebral disk with its soft nucleus and the adjacent, hard vertebral bodies. If one of the end plates is destroyed, nucleus tissue can penetrate into the vertebral body so that the nucleus can no longer carry out its function as a pressure cushion. A break in the intervertebral disk can in turn cause the running out of the nucleus pulposus through a fracture in the anulus fibrosus, which results in a pressing against the ganglions or against the spinal nerve and causes severe pain both in the back and in the legs.

For the treatment of intervertebral disk damage, depending on the degree of damage, the affected intervertebral disk is completely removed and is replaced by an implant, the nucleus is completely removed from the intervertebral disk and is replaced by a corresponding implant called a nucleus replacement or a part of the nucleus pulposus is removed and is replaced by an implant called a cage. Each of the aforesaid implants usually consists of a hollow body made of a textile material and filled with a filler material. Such an implant, which consists of a high-tensile wire fabric or fiber fabric compatible with the human body tissue, for example of a titanium fabric, and can be filled with a suitable fluid as a filler material, is known, for example, from EP 0 480 954 B1.

An implant is known from US 2002/0029083 A1 for an artificial intervertebral disk which includes a layer of a keratin material, for example of a keratin hydrogel, and optionally a further layer of a polymer, for example polyethylene, polytetrafluoroethylene, polystyrene or the like.

Another important application of textile materials in the medical field is represented by hoses with which inter alia viscous liquids are introduced into the human body. An example for this is represented by hoses via which filler material can be introduced into implants already introduced into the body. A further example for this is vertebroplasty for the treatment of osteoporosis. In this process, viscous bone cement is injected via a hose connected to a cannula at one end into the porous bone to fill the bone with the bone cement for inner stabilization. For this purpose, due to the high viscosity of the bone cement, hoses having a sufficiently large internal diameter have to be used to ensure a sufficient conveying of the bone cement through the hose. In addition, the hoses must have a sufficient impermeability with respect to the material to be conveyed to avoid a passage of the material to be conveyed, for example bone cement, from the hose into the inside of the body. The currently known textile materials from which such hoses are manufactured are, however, not sufficiently impermeable for the materials to be conveyed. The sliding properties of the known textile materials are also in need of improvement.

In accordance with an aspect of the present disclosure, a textile material should be provided which has sufficient sliding properties and is sufficiently impermeable in particular with respect to filler materials usually used in intervertebral disk implants or vertebral body implants and can therefore advantageously be used in the medical field as a hose material, for example for vertebroplasty, or as an implant material, for example for nucleus replacement. In addition, the textile material as well as its coating should have sufficient biostability.

In accordance with an aspect of the present disclosure, a textile material comprising a textile substrate is provided, with a coating being provided at least on a part of the surface of the textile substrate, said coating including a compound selected from the group consisting of hydrogels, of polyurethanes, polyvinyl chloride, polytetrafluoroethylene and any desired combinations of two or more of the aforesaid compounds or consists of one of the aforesaid compounds.

Surprisingly, it was able to be found within the framework of the present invention that textile substrates coated with a compound selected from the group consisting of hydrogels with the exception of keratin hydrogels, from polyurethanes, polyvinyl chloride, polytetrafluoroethylene and any desired combinations of two or more of the aforesaid compounds have an exceptional permeability with respect to filler materials, for example bone cement, used in the medical field and are additionally characterized by good sliding properties. Furthermore, these materials have exceptional biostabflity as well as a reproducible quality so that the effort and/or expense for the quality control in the manufacture of these materials can be kept within limits. For the aforesaid reasons, the textile materials can advantageously be used as a material for implants, for example an artificial intervertebral disk, a replacement of the nucleus pulposus, cage vertebral body implant or a joint surface replacement or as medical hose material, for example for vertebroplasty.

A hydrogel in the sense of the present invention is understood in agreement with the relevant technical literature as a polymer containing water, but water insoluble, whose molecules are chemically or physically linked to a three-dimensional network.

Generally, a part of the total surface or the total surface of the textile substrate can be coated with the material previously described. For this purpose, it is also possible to coat the individual fibers of the textile substrate or at least some of the fibers of the textile substrate accordingly. For this reason, a textile material including a textile substrate is proposed in which the textile substrate consists of fibers and at least some of the fibers are provided with a coating which includes a compound selected from the group consisting of synthetic hydrogels, polyurethanes, polyvinyl chloride, polytetrafluoroethylene and any desired combinations of two or more of the aforesaid compounds or consists of one of the aforesaid compounds. Due to the use of synthetic coating materials, the corresponding textile materials have exceptional biocompatibility as well as a reproducible quality so that the effort and/or expense for the quality control in the manufacture of the corresponding materials can be kept within limits.

When a synthetic hydrogel is used, the present invention is not limited with respect to the chemical nature of the synthetic hydrogel or with respect to the number of the hydrogels contained in the coating. In this respect, the coating of the textile substrate can include a synthetic hydrogel or at least two different synthetic hydrogels, with the coating being able to include other auxiliary materials and additives in addition to the at least one synthetic hydrogel. Particularly good results are obtained when the coating of the textile substrate consists of one or of at least two different synthetic hydrogels. Such materials have a particularly high non-permeabflity with respect to filler materials usually used in the medical field, for example bone cement, as well as good biostability.

In a further development of the idea of the invention, it is proposed that the coating of the textile substrate include a hydrogel based on a vinyl polymer or in particular consists of a hydrogel based on vinyl polymer.

Basically, hydrogels from all vinyl polymers known to the skilled person for this purpose can be used for the coating of the textile substrate, with in particular vinyl polymers having proved to be suitable for this purpose which are selected from the group consisting of polyvinyl alcohol, polyvinyl acetate, polyvinyl butyral, polyvinyl pyrrolidon and any desired mixtures of at least two of the aforesaid compounds. Particularly good results are obtained when the coating of the textile substrate contains a polyvinyl alcohol hydrogel or in particular when the coating of the textile substrate consists of a polyvinyl alcohol hydrogel.

In accordance with an exemplary embodiment, the coating of the textile material includes a polyurethane and in particular a polycarbonate urethane. The polycarbonate urethane can be made up both of aromatic hard segments and of aliphatic hard segments. A polycarbonate urethane having silicone end groups is equally possible. Particularly good results are in particular obtained when the coating consists of a polyurethane or of a polycarbonate urethane. In particular polycarbonate urethanes are characterized by exceptional biostability so that they are particularly suitable as coating material.

Polycarbonate urethanes have provided to be particularly suitable as coating materials which have a weight averaged molecular weight from 60,000 to 500,000 g/mol and in particular from 90,000 to 400,000 g/mol, where the ratio between the hard segments and the soft segments should be between 20%/80% and 60%/40%.

The thickness of the coating provided on the textile substrate of the textile material or of the coating provided on the individual fibers or on some of the fibers of the textile substrate in particular depends on the later purpose of the textile material. Good results are in particular obtained with textile materials which have a coating thickness between 0.5 and 200 μm. In accordance with a further exemplary embodiment of the present disclosure, the thickness of the coating amounts to between 0.5 and 50 μm and in particular between 0.5 and 10 μm. in addition to sufficient sliding properties, such coatings also have a high non-permeability with respect to filler materials usually used in the medical sector. With too low a layer thickness, a sufficient non-permeability of the textile material with respect to the filler material cannot be achieved, whereas with too large a layer thickness there is the risk that the coating separates completely or at least partly from the substrate over time.

In particular when the textile material should be used in the medical field, it has proven to be advantageous for the textile substrate to consist of biocompatible and/or resorbable material. This includes both the case that the textile substrate consists of an individual biocompatible material and/or resorbable material and the case that it consists of a mixture of at least two different materials which are each biocompatible and/or resorbable.

The textile substrate can generally be used in the form of a textile composite, that is of a textile material which is neither woven, nor knitted nor fabric woven, and/or in the form of a woven product, a knitted product or a fabric woven product. Examples for suitable textile composites are felts or fleece materials. Good results are in particular obtained when a textile substrate is provided in the textile material and consists of a fabric, a knitted fabric or a woven fabric made of textile fibers.

Particularly good results are in particular obtained when the textile substrate consists of a fabric, of a knitted fabric or of a woven fabric of textile fibers which is sealed in a liquid tight manner by the coating.

The material of the textile fibers forming the textile substrate can be freely selected in dependence on the designated purpose of the material. It has in particular proven to be advantageous with a designated use as a hose material or as an implant material for the textile substrate of the textile material to include textile fibers made of a compound selected from the group consisting of polyethylene terephthalate (PET), polyetherketones (PEEK), polymethyl methacrylates (PMMA), titanium (Ti), cobalt/chromium alloys (CoCr), hydrogels with the exception of keratin hydrogels, polyvinyl alcohol (PVA), polyolefines, in particular polyethylene (PE) and polypropylene (PP) and any desired combinations of two or more of the aforesaid compounds or consists of textile fibers of one of the aforesaid compounds. Such materials are in particular characterized by exceptional biocompatibility. PET is in particular suitable for this purpose.

A further subject of the present disclosure is a method for the manufacture of a textile material, in particular of a previously described textile material, wherein a textile substrate is coated at least partly with a compound selected from the group consisting of hydrogels with the exception of keratin hydrogels, of polyurethanes, in particular polycarbonate urethanes, of polyvinyl chloride, polytetrafluoroethylene and any desired combinations of two or more of the aforesaid compounds. This includes the case that the total surface or the total surfaces of the textile substrate are coated with one of the aforesaid compounds and also the case that only a part of the surface of the textile material is coated with such a compound. In the latter case, it can be a contiguous, correspondingly coated part of the surface of the textile substrate or a plurality of part regions of the surface of the textile substrate spatially separated from one another.

The method is generally not restricted to individual coating techniques. It is rather the case that the application of the coating to the textile substrate can take place with any technique known to the skilled person for this purpose.

The coating of the textile substrate can in particular take place, for example, by dipping the textile substrate into a synthetic hydrogel solutions and a subsequent subjecting of the substrate provided with the hydrogel solution to at least one freeze-thaw cycle when the coating includes or consists of a hydrogel with the exception of keratin hydrogel. This method has in particular proved to be advantageous when the total surface or the total surfaces of the textile substrate should be coated with a hydrogel. However, the method explained above is also suitable for only a partial coating of the textile substrate, with the parts of the surface not to be coated being correspondingly covered in this case. In a provided one-sided coating of the textile substrate, the textile substrate can, for example, be applied to a carrier material and can be dipped with this carrier material into the hydrogel solution, with only the surface of the textile substrate disposed opposite the carrier people being coated with the hydrogel.

In the case of the coating of the textile substrate with a polyvinyl alcohol hydrogel, for example, the hydrogel solution into which the textile substrate to be coated is dipped can, for example, be an aqueous solution consisting of polyvinyl alcohol hydrolyzed to a strength 2 to 40% by weight, in particular of 5 to 20% by weight. This solution can be manufactured, for example, in that a corresponding amount of polyvinyl alcohol is dissolved in a corresponding amount of water at 95° C.

The present invention is also not restricted with respect to the number of freeze-thaw cycles to be carried out. It has, however, proven to be advantageous to subject the textile substrate to be coated to at least two freeze-thaw cycles and in particular to precisely two freeze-thaw cycles. In each of the individual freeze-thaw cycles, the coated substrate is first incubated for a sufficient time period at temperatures of less than 0° C. to freeze the material. The temperature of the freeze stage of the freeze-thaw cycle, for example, amounts to between −1 and −50° C. and in particular between −10 and −20° C. and the time period for the freeze stage of the freeze-thaw cycle amounts to at least 8 hours. The purpose of the freeze-thaw cycle treatment is the achievement of crosslinks between the individual molecules of the hydrogel to increase the durability of the hydrogel.

A further subject matter of the present invention is an article, in particular an implant, having an outer envelope, with the outer envelope consisting of the previously described textile material. Due to the coating of the textile substrate of the textile material with a compound selected from the group consisting of hydrogels with the exception of keratin hydrogels, of polyurethanes, in particular polycarbonate urethanes, of polyvinyl chloride, polytetrafluorotheylene and any desired combinations of two or more of the aforesaid compounds, such an article is characterized by a high non-permeability for filler materials usually used in the medical field and additionally by good sliding properties and exceptional biostability.

In accordance with a possible embodiment of the present invention, the article is made as a hose. Due to the high non-permeability of the textile substrate coated with a hydrogel and to its good biocompatibility, such a hose is exceptionally suitable for the introduction of liquids, in particular high-viscosity liquids, into the human body, such as the introduction of bone cement in vertebroplasty. Alternatively, the article can, for example, also be made as a ring or in balloon shape.

Generally, the hose, ring or balloon can be coated at both sides or at one side with at least one hydrogel. It has in particular proven to be advantageous when the hose is provided for the introduction of liquid into a human body to coat the hose at both sides or to coat at least the inner side of the article with the aforesaid coating, in particular with a hydrogel.

In accordance with a further possible embodiment of the present disclosure, a filler material is contained in the envelope of the article which is surrounded, in particular at all sides, by the envelope.

The filler material can, for example, be bone cement, a synthetic hydrogel and/or Ringer's solution, with the present invention not being limited to one of the aforesaid materials.

Due to its properties, the article—for example, filled with bone cement, a synthetic hydrogel and/or Ringer's solutions—can be used as an artificial intervertebral disk, a replacement for the nucleus pulposus, a cage vertebral body implant or as a joint surface replacement.

Alternatively to this, the article—made as a hose—can be used in vertebroplasty or in nucleoplasty.

The object disclosed in the present case will be described in the following purely by way of example with reference to advantageous embodiments and to the enclosed drawings. There are shown:

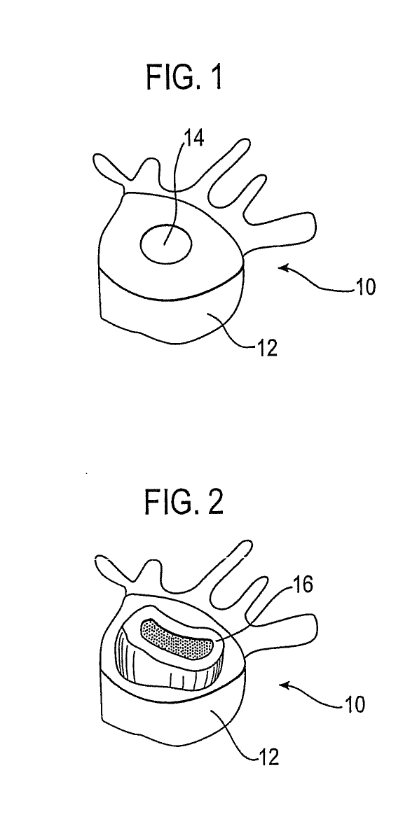

FIG. 1 a schematic perspective view of a natural human intervertebral disk;

FIG. 2 a schematic perspective view of an intervertebral disk with an implant in accordance with a first embodiment of the present invention;

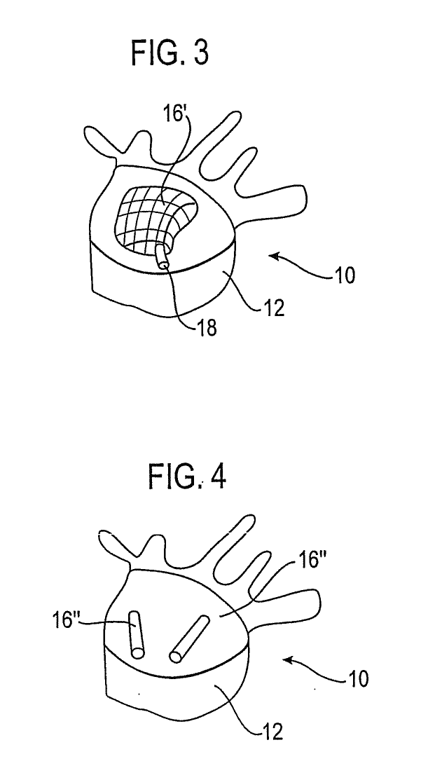

FIG. 3 a schematic perspective view of an intervertebral disk with an implant in accordance with a second embodiment of the present invention;

FIG. 4 a schematic perspective view of an intervertebral disk with an implant in accordance with a third embodiment of the present invention;

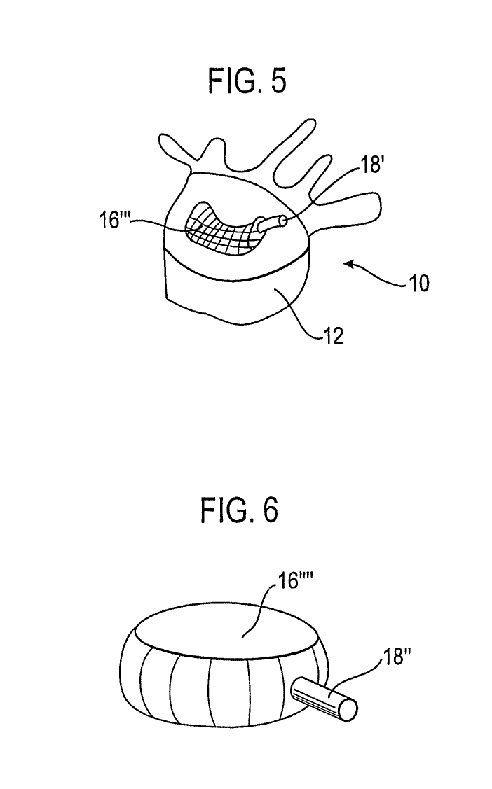

FIG. 5 a schematic perspective view of an intervertebral disk with an implant in accordance with a fourth embodiment of the present invention;

FIG. 6 a schematic perspective view of an implant in accordance with a fifth embodiment of the present invention;

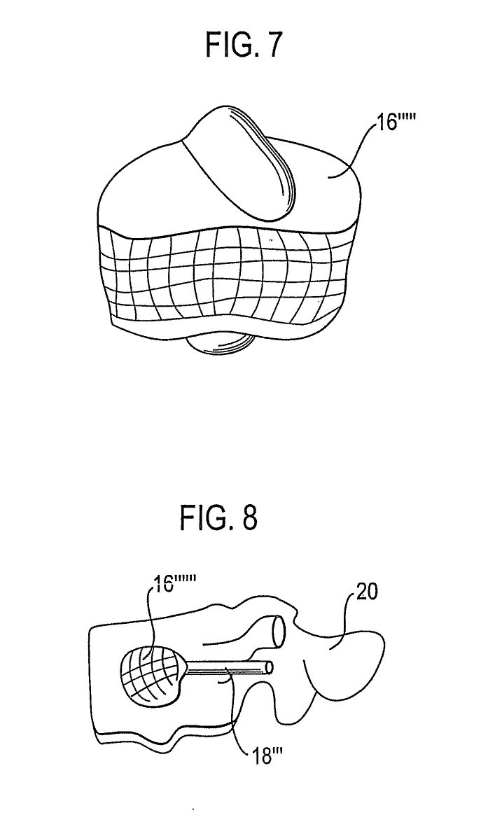

FIG. 7 a schematic perspective view of an implant in accordance with a sixth embodiment of the present invention;

FIG. 8 a schematic perspective view of a vertebral body with an implant in accordance with a seventh embodiment of the present invention;

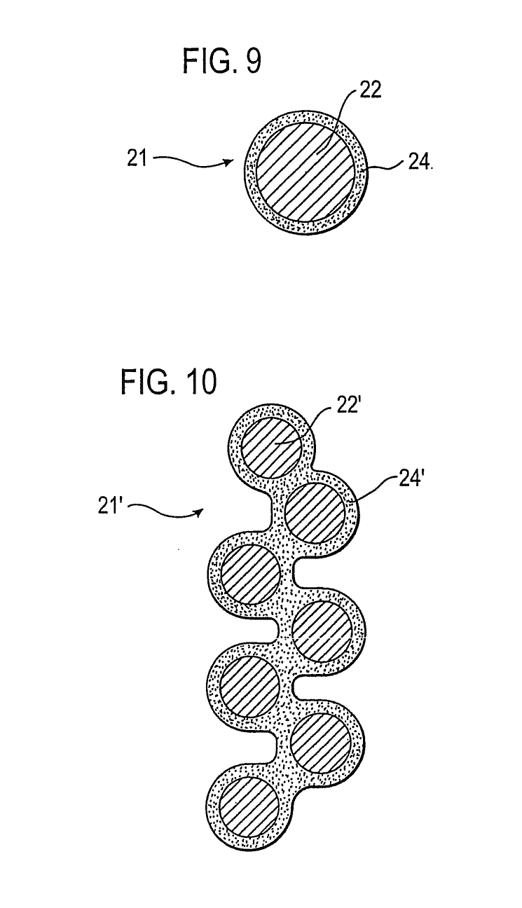

FIG. 9 a cross-sectional view of a textile material in accordance with a first embodiment of the present invention; and

FIG. 10 a cross-sectional view of a textile material in accordance with a second embodiment of the present invention.

The intervertebral disk 10 shown schematically in FIG. 1 has an anulus fibrosus 12 which is composed of highly structured collagen fibers embedded into an amorphous base substance. A hollow space in which the nucleus of the intervertebral disk (nucleus pulposus) 14 is located is at the center of the annulus fibrosus 12. The soft nucleus takes up approximately 25 to 40% of the total cross-section of the disk and is mainly composed of mucoid material which mainly includes proteoglycanes having a low portion of collagen. A respective vertebral end plate (not shown) is provided above and beneath the intervertebral disk 10 in the region of the nucleus pulposus, said end plates being composed of hyaline cartilage mass and separating the intervertebral disk 10 from the adjacent vertebral bodies (not shown).

The nucleus pulposus has been removed in the intervertebral disk shown in FIG. 2 and has been replaced by an implant 16 substantially made in ring shape. The interior of the implant 16 has been filled with bone or with bone replacement material. Such implants, which only replace part of the nucleus, are generally also called cages.

An intervertebral disk 10 having a bag-shaped implant 16′ is shown in FIG. 3. In this case, the total nucleus was removed from the intervertebral disk 10 and replaced by the implant 16′. As can be seen from FIG. 3, the implant 16′ has a hose piece 18 through which the filler material for the implant 16′ can be introduced. This filler material can, for example, be bone cement, a hydrogel and/or Ringer's solution. The introduction of the filler material through the hose piece 18 into the implant 16′ can take place before or also after the insertion of the implant 16′ into the patient.

Whereas the cage shown in FIG. 4 has two bar-shaped implants 16″, the nucleus replacement shown in FIG. 5 consists of a hose-shaped implant 16′″ with a hose piece 18′.

In FIG. 6, an implant 16″″ is shown which is likewise of bag shape and whose outer envelope consists of the textile material with one of the aforesaid coating materials, in particular a hydrogel coating. This implant 16″″ is made such that it can completely replace an intervertebral disk (artificial intervertebral disk). This implant 16″″ also has a hose piece 18″ through which the filler material, for example Ringer's solution, hydrogel and/or bone cement, can be introduced into the implant 16″″.

The implant 16′″″ shown in FIG. 7 and suitable as an artificial intervertebral disk has a body whose cross-section is shaped in accordance with the natural intervertebral disk and on which two elevated portions simulating an end plate are provided or which is connected in each case at its upper side and its lower side to a respective end plate.

In FIG. 8, a vertebral body 20 is shown schematically into which an implant 16″″″ is inserted. The outer enveloped of this implant 16″″″ also consists of textile material coated with hydrogel and is filled with a filler material via a hose piece 18′″. Such an implant 16″″″ can be used, for example, in the case of a fracture of the vertebral body, whether caused by a trauma or by osteoporosis.

Whereas the textile material 21 shown in FIG. 9 consists of a fiber 22 having a substantially circular cross-section and of a coating 24 applied to the fiber surface, the textile material 21′ shown in FIG. 10 includes a plurality of fibers 22′, with the total fiber structure being provided with a coating 24′.

The present invention will be explained in the following by an example illustrating it, but not restricting it.

### Example

Two hoses having an internal diameter of 4 mm each and a length of 100 mm each were manufactured from woven PET fibers such as are used for the manufacture of cords which are used in the dynamic spinal column stabilization system Dynesys®. For the purpose of coating, these hoses were subsequently dipped into a solution which comprises polyvinyl alcohol hydrolyzed to a strength of 10% by weight and which was manufactured by dissolving the corresponding amount of polyvinyl alcohol into water at 95° C. to coat the hoses with polyvinyl alcohol both at their inner surfaces and at the outer surfaces of the textile substrate.

The hoses coated in this manner were thereupon subjected to two freeze-thaw cycles in that the hoses were first incubated for 3 days at −17° C. and were subsequently incubated for 5 hours at room temperature and were thereby thawed before the hoses were again frozen for 12 hours at −17° C. and subsequently incubated for 2 hours at room temperature. The hoses thus coated with hydrogel were subsequently treated in water for 2 hours.

Each of the two hoses manufactured in this way were mechanically fastened to the tip of a 60 ml syringe.

Subsequently, bone cement based on PMMA without zirconium dioxide (SULCEM 3®) was introduced into each of the two hoses via the syringe. The bone cement was mixed for 60 seconds before introduction into the syringe and incubated for a further 30 seconds to remove air bubbles. Subsequently, the cement was filled into the syringe and conveyed under pressure through the syringe and the hose.

The bone cement could be conveyed through both hoses without problem without bone cement being forced through the hose material to the outside.

This example shows that the textile material has exceptional non-permeability with respect to filler materials such as bone cement usually used in the medical field and has sufficient sliding properties to convey even high-viscosity materials through it.

### Comparison Example

The same procedure as in Example 1 was followed, with the exception that instead of two hoses coated with polyvinyl alcohol hydrogel, two corresponding hoses made of woven PET fibers, but uncoated, were used.

It was impossible to convey bone cement through the uncoated hoses; it was rather the case that the bone cement flow stopped after 16 and 18 m respectively. No further conveying could be achieved even by increasing the force effective at the syringe.

### REFERENCE NUMERAL LIST

10 intervertebral disk

12 fibrocartilage ring (anulus fibrosus)

14 nucleus (nucleus pulposus)

16 implant (cage)

16′ implant (replacement of the nucleous pulposus)

16″ implant (cage)

16′″ implant (replacement of the nucleous pulposus)

16″″ implant (artificial intervertebral disk)

16′″″ implant (artificial intervertebral disk)

16″″″ implant (vertebral body implant)

18 to 18′″ hose (piece)

20 vertebral body

21, 21′ textile material

22, 22′ fiber/substrate

24, 24′ coating

1. A textile material comprising a textile substrate, wherein a coating is provided at at least one part of the surface of the textile substrate, wherein the textile substrate is made of fibers and at least some of the fibers are provided with the coating which includes a compound selected from the group consisting of synthetic hydrogels, polyurethanes, polyvinyl chloride, polytetrafluoroethylene and any desired combinations of two or more of the aforesaid compounds.

2. A textile material in accordance with claim 1, wherein the coating is made of a synthetic hydrogel or at least two different synthetic hydrogels.

3. A textile material in accordance with claim 2, wherein the coating includes or is made of a vinyl polymer hydrogel.

4. (canceled)

5. A textile material in accordance with claim 1, wherein the coating is made of a polyurethane.

6-7. (canceled)

8. A textile material in accordance with claim 5, wherein the coating has a thickness of 0.5 to 200 μm.

9. A textile material in accordance with claim 8, wherein the textile substrate is made of biocompatible material and/or of resorbable material.

10. A textile material in accordance with claim 9, wherein the textile substrate is a fabric made of textile fibers wherein the textile fibers include a compound selected from the group consisting of polyethylene terepththalate, polyether ketones, polymethyl methacrylates, titanium, cobalt/chromium alloys, hydrogels, polyvinyl alcohol, polyolefines, and mixtures of the aforesaid compounds.

11-13. (canceled)

14. A method for the manufacture of textile material, wherein a textile substrate of fibers is at least partly coated with a compound selected from the group consisting of synthetic hydrogels, polyurethanes, polycarbonate urethanes, polyvinyl chloride, polytetrafluorethylene and any desired combinations of two or more of the aforesaid compounds.

15. A method in accordance with claim 14, wherein the textile substrate is dipped into a hydrogel solution and is subsequently subjected to at least one freeze-thaw cycle.

16. (canceled)

17. An article having an outer envelope, wherein the outer envelope comprises a textile material 15 in accordance with claim 1.

18. An article in accordance with claim 17, wherein the textile material is made as a hose, as a ring or in balloon shape.

19. An article in accordance with claim 18, wherein the hose, ring or balloon is coated on both sides or on one side.

20. An article in accordance with claim 17, wherein a filler material is included in the envelope which is surrounded by the envelope, wherein said filler material is bone cement, a hydrogel and/or Ringer's solution.

21. (canceled)

22. Use of an article in accordance with claim 20 as an artificial intervertebral disk, as a replacement for the nucleus pulposus, a cage, a vertebral body implant or a joint surface replacement for vertebral body fusion or for vertebral body reconstruction.

23. Use of an article in accordance with claim 18 in vertebroplasty or nucleoplasty.

24. A textile material in accordance with claim 1, wherein the coating is made of a polycarbonate urethane.

25. A textile material in accordance with claim 1, wherein the coating has a thickness of 0.5 to 50 μm.

26. A textile material in accordance with claim 1, wherein the coating has a thickness of 0.5 to 10 μm.

27. A textile material in accordance with claim 9, wherein the textile substrate is a fabric made of textile fibers wherein the textile fibers include a compound selected from the group consisting of polyethylene terepththalate, polyether ketones, polymethyl methacrylates, titanium, cobalt/chromium alloys, hydrogels, polyvinyl alcohol, polyethylene, polypropylene, and mixtures of the aforesaid compounds.

28. A method for treating intervertebral disk damage comprising implanting in a patient in need of such treatment an article according to claim 20.
